(19) United States
(12) Patent Application Publication
(10) Pub. No.: US 2023/0000827 A1
(43) Pub. Date: Jan. 5, 2023

(54) INHIBITORS OF HSP70 PROTEIN

(71) Applicants: INSERM (INSTITUTE NATIONAL DE LA SANTÉ ET DE LA RECHERCHE MÉDICALE), Paris (FR); UNIVERSITE DE BOURGOGNE, Dijon (FR); CENTRE HOSPITALIER UNIVERSITAIRE DIJON BOURGOGNE, Dijon (FR)

(72) Inventors: Carmen GARRIDO FLEURY, Dijon (FR); Frédéric LIRUSSI, Dijon (FR)

(21) Appl. No.: 17/776,415

(22) PCT Filed: Nov. 19, 2020
(86) PCT No.: PCT/EP2020/082648

§ 371 Date: May 12, 2022

(30) Foreign Application Priority Data
Nov. 20, 2019 (EP) 19306494.6

(51) Int. Cl. A61K 31/41; A61K 31/454; A61P 35/00; A61K 45/06

(52) U.S. Cl. CPC A61K 31/41; A61K 31/454; A61P 35/00; A61K 45/06

(57) ABSTRACT

This invention relates to compounds that are inhibitors of HSP70 protein, and applications thereof.

### TECHNICAL FIELD OF THE INVENTION

This invention relates to inhibitors of HSP70 protein, and their use for cancer treatment.

### BACKGROUND OF THE INVENTION

Apoptosis is a cell death process frequently observed in cancer cell after treatment with anti-cancer agents. In mammals, apoptosis involves mitochondrial proteins such as cytochrome c (Liu X, et al. Cell. 1996;86:147-157) and apoptosis-Inducing Factor (AIF) (Ferri K F, et al. Nat Cell Biol. 2001;3:E255-263). These molecules can be released from the mitochondrial intermembrane space to the cytosol of stressed or injured cells. Once in the cytosol, cytochrome c interacts with the adaptator molecule apoptotic protease activation factor-1 (Apaf-1) to trigger its ATP-dependent oligomerization (Hu Y. et al. Embo J. 1999;18:3586-3595; Li P et al. Cell. 1997;91:479-489) that allows the apoptosome complex formation (Zou H et al. J Biol Chem. 1999;274:11549-11556; Saleh A et al. J Biol Chem. 1999;274:17941-17945). Then, caspase-9 is activated by the apoptosome, setting on a caspase cascade that leads to apoptosis (Li P, et al. Cell. 1997;91:479-489). Unlike cytochrome c, AIF directly migrates to the nucleus to induce DNA fragmentation and a caspase-independent apoptosis (Daugas E et al. Faseb J. 2000;14:729-739; Susin S A, et al. Nature. 1999;397:441-446).

The inducible heat shock protein 70 (HSP70) is an evolutionary conserved protein whose expression enhances the ability of the cell to survive to a panoply of lethal conditions. It is an ATP-dependent molecular chaperone that assists the folding of newly synthesized polypeptides, the assembly of multi-protein complexes and the transport of proteins across cellular membranes (Beckmann R P et al. Science. 1990;248:850-854; Murakami H et al. J Cell Biol. 1988;107:2051-2057; Shi Y et al. Mol Cell Biol. 1992;12:2186-2192).

Under normal cell growth, HSP70 is no or very little expressed. In contrast, under stressful conditions, HSP70 expression is transiently induced. Elevated HSP70 levels allow the cells to survive the stressful situation. Part of HSP70 protective function is associated to its anti-apoptotic properties. HSP70 is a powerful anti-apoptotic protein able to block caspase-dependent apoptosis by its interaction with Apaf-1, and caspase independent apoptosis by its association with AIF. HSP70 is also able to block the release of cathepsines from the lysosomes thereby also blocking cell death. Confirming the important protective function of HSP70, cells lacking hsp70.1 and hsp70.3, the two genes that code for inducible HSP70, are highly sensitive to apoptosis induced by wide range of lethal stimuli (Schmitt E et al. Cancer Res. 2003;63:8233-8240).

HSP70 is often constitutively expressed in human tumor samples from various origins, and its expression may further increase after chemotherapy (Parcellier A et al. Biochem Biophys Res Commun. 2003;304:505-512). This overexpression of HSP70 is needed for the cancer cell survival. Overexpressed HSP70 in human cancer cells is associated with metastasis, poor prognosis and resistance to chemotherapy or radiation therapy (Conroy SE et al. Br J Cancer. 1996;74:717-721; Fuller K J, et al. Eur J Cancer. 1994;30A:1884-1891; Brondani Da Rocha A, et al. Int J Oncol. 2004;25:777-785; Vargas-Roig L M et al. Int J Cancer. 1998;79:468-475; Nanbu K et al. Cancer Detect Prey. 1998;22:549-555). The inventors and other group have previously shown that down-regulation of HSP70 by mean of an antisense construction or a siRNA, reduces cancer cell tumorigenicity in rodent models (Gurbuxani S. et al. Oncogene. 2001;20:7478-7485; Nylandsted J. et al. Proc Natl Acad Sci USA. 2000;97:7871-7876).

A construct derived from AIF, ADD70 (AIF-Derived Decoy for HSP70), that inhibits and neutralizes HSP70 in the cytosol, was shown to allow cancer cell sensitization to anticancer drugs such as cisplatin or etoposide (Schmitt E, et al. Cancer Res. 2003;63:8233-8240). In vivo, ADD70 treatment in murine cancer models reduces tumor growth and even induces tumor regression. These effects are increased with a single cisplatin injection in animals (Schmitt E et al. Cancer Res. 2006;66:4191-4197).

However, ADD70 is too big to serve as a biotherapeutic molecule. Thus, it remains a need to develop small molecules able to inhibit HSP70 for the treatment of tumor.

### SUMMARY OF THE INVENTION

The inventors characterized different aptamers which specifically block the HSP70 chaperone activity and induce tumor regression. The tumor regression was associated with an increase in tumor cells apoptosis and surprisingly by a spectacular increase in the amount of cytotoxic (ROS-producers) macrophages infiltrating the tumor. Thus, the molecules developed by the inventors targeting specifically HSP70 could constitute a new a type of immunotherapeutic molecule against cancer.

A first aspect of the present invention relates to a compound of formula (I) or (II) for its use in the treatment of tumors and preferably solid tumors of a patient by inhibiting the HSP70 protein activity thereby inducing apoptosis of tumor cells and re-educating macrophages

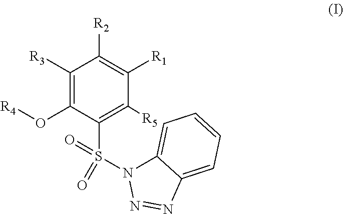

wherein R₁ represents a hydrogen atom, an halogen atom, a C1-C8 alkyl group, preferably a C1-C3 alkyl group,

R₂ represents a hydrogen atom, an halogen atom, a C1-C8 alkyl group, preferably a C1-C3 alkyl group,

R₃ represents a hydrogen atom, an halogen atom, a C1-C8 alkyl group, preferably a C1-C3 alkyl group,

R₄ represents a C1-C8 alkyl group, preferably a C1-C3 alkyl group, and

R₅ represents a hydrogen atom, an halogen atom, a C1-C8 alkyl group, preferably a C1-C3 alkyl group;

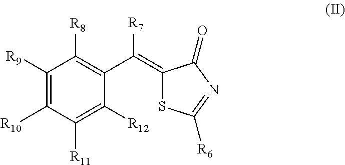

wherein R₆ represents an heterocycle preferably a piperidinyl group,

R₇ represents a hydrogen atom or a halogen atom,

R₈ represents a hydrogen atom or a halogen atom,

R₉ represents a hydrogen atom or a halogen atom,

R₁₀ represents a hydrogen atom or a halogen atom,

R₁₁ represents a hydrogen atom or a halogen atom, and

R₁₂ represents a hydrogen atom or a halogen atom.

A second aspect of the present invention relates to a complex formed between a compound of formula (I) or (II) and a lipoprotein, preferably a High Density Lipoprotein (HDL),

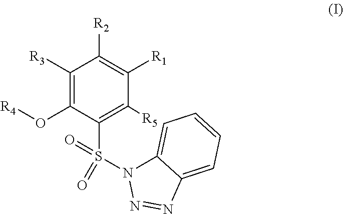

wherein R₁ represents a hydrogen atom, an halogen atom, a C1-C8 alkyl group, preferably a C1-C3 alkyl group,

R₂ represents a hydrogen atom, an halogen atom, a C1-C8 alkyl group, preferably a C1-C3 alkyl group,

R₃ represents a hydrogen atom, an halogen atom, a C1-C8 alkyl group, preferably a C1-C3 alkyl group,

R₄ represents a C1-C8 alkyl group, preferably a C1-C3 alkyl group, and

R₅ represents a hydrogen atom, an halogen atom, a C1-C8 alkyl group, preferably a C1-C3 alkyl group;

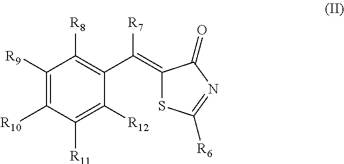

wherein R₆ represents an heterocycle preferably a piperidinyl group,

R₇ represents a hydrogen atom or a halogen atom,

R₈ represents a hydrogen atom or a halogen atom,

R₉ represents a hydrogen atom or a halogen atom,

R₁₀ represents a hydrogen atom or a halogen atom,

R₁₁ represents a hydrogen atom or a halogen atom, and

R₁₂ represents a hydrogen atom or a halogen atom.

### DETAILED DESCRIPTION OF THE PREFERRED EMBODIMENTS

The inventors characterized different aptamers which specifically block the HSP70 chaperone activity and induce tumor regression.

A first aspect of the present invention relates to a compound of formula (I) or (II) for its use in the treatment of tumors and preferably solid tumors of a patient by inhibiting the HSP70 protein activity thereby inducing apoptosis of tumor cells and re-educating macrophages.

The term “halogen atom” used in the present invention refers to fluorine, chlorine, bromine or iodine atoms.

The term “heterocycle” used in the present invention refers to a pyridinyl group, a piperidinyl group, preferably to a piperidinyl group.

### Compounds

Compound of Formula (I)

The compound of formula (I) of the present invention is represented by formula (I)

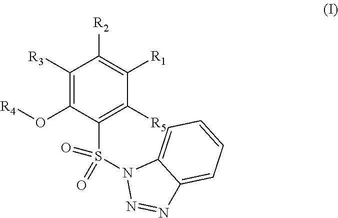

wherein R₁ represents a hydrogen atom, an halogen atom, a C₁-C₈ alkyl group, preferably a C₁-C₃ alkyl group,

R₂ represents a hydrogen atom, an halogen atom, a C₁-C₈ alkyl group, preferably a C₁-C₃ alkyl group,

R₃ represents a hydrogen atom, an halogen atom, a C₁-C₈ alkyl group, preferably a C₁-C₃ alkyl group,

R₄ represents a C₁-C₈ alkyl group, preferably a C₁-C₃ alkyl group, and

R₅ represents a hydrogen atom, an halogen atom, a C₁-C₈ alkyl group, preferably a C₁-C₃ alkyl group.

In a particular embodiment, the compound of formula (I) is chosen from the group consisting

of a compound of formula (B)

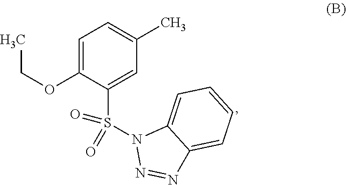 [SMILES: CCOc1ccc(C)cc1S(=O)(=O)n1nnc2ccccc21]

and a compound of formula (C)

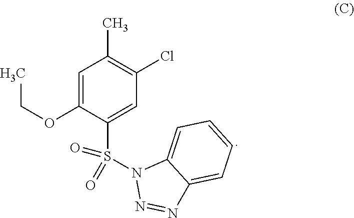 [SMILES: CCOc1cc(C)c(Cl)cc1S(=O)(=O)n1nnc2ccccc21]

Compound of formula (II)

The compound of formula (II) of the present invention is represented by formula (II)

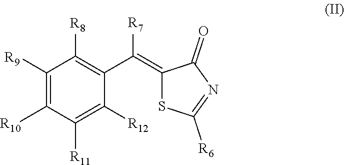

wherein R₆ represents an heterocycle preferably a piperidinyl group,

R₇ represents a hydrogen atom or a halogen atom,

R₈ represents a hydrogen atom or a halogen atom,

R₉ represents a hydrogen atom or a halogen atom,

R₁₀ represents a hydrogen atom or a halogen atom,

R₁₁ represents a hydrogen atom or a halogen atom, and

R₁₂ represents a hydrogen atom or a halogen atom.

In a particular embodiment, the compound of formula (II) is a compound of formula (A)

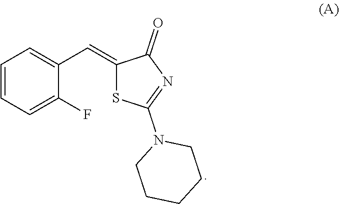 [SMILES: O=C1N=C(N2CCCCC2)S/C1=C\c1ccccc1F]

Synthesis

Compounds of formula (I) and of formula (II) are commercially available.

Compounds of formula (I) can be synthetized according to the following protocol (scheme 1):

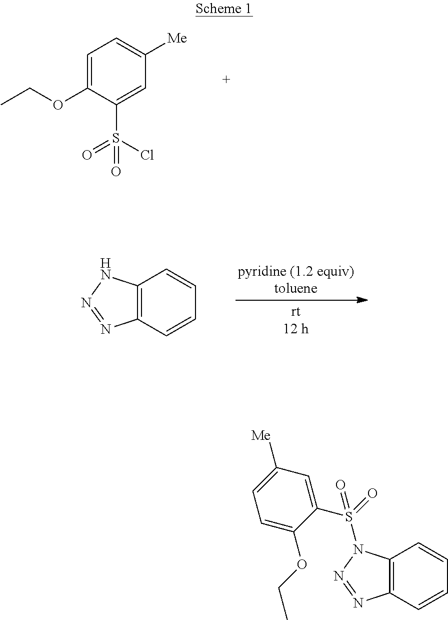 [SMILES: CCOc1ccc(C)cc1S(=O)(=O)Cl ; c1ccc2[nH]nnc2c1 ; CCOc1ccc(C)cc1S(=O)(=O)n1nnc2ccccc21]

To an ice-cold solution of benzotriazole (0.01 mol) and pyridine (0.016 mol) in dry toluene (12 mL) was added dropwise 2-Ethoxy-5-methylbenzene-1-sulfonyl chlorid (CAS: 187471-28-9) (0.012 mol) in toluene (3 mL). The mixture was then stirred overnight at room temperature. AcOEt (15 mL) and H₂O(10 mL) were added. The organic layer was separated, successively washed with water and brine, and dried over anhydrous MgSO₄. Removal of solvents in vacuo gave a crude which was purified through flash chromatography.

The skilled person will be able to adjust the process with well-known steps in order to synthetize all the compounds covered by formula (I).

Complex

To increase the solubility and favour cellular uptake, the compound of formula (I) or (II) as previously described can be complexed into a lipoprotein.

Thus, a second aspect of the present invention relates to a complex formed between the compound of formula (I) or (II) as previously described and a lipoprotein.

“Lipoproteins” refers to complex particles that have a central hydrophobic core of non-polar lipids, primarily cholesterol esters and triglycerides. This hydrophobic core is surrounded by a hydrophilic layer consisting of phospholipids, free cholesterol, and apolipoproteins. Plasma lipoproteins are divided into several classes based on size, lipid composition, and apolipoprotein composition (chylomicrons, chylomicrons remnants, very low-density lipoprotein (VLDL), intermediate density lipoprotein (IDL), low density lipoprotein (LDL), high density lipoprotein (HDL) and Lp(a)).

In particular, the lipoprotein can be a high density lipoprotein (HDL) or low density lipoprotein (LDL) well-known in the skilled person in the art (Introduction to Lipids and Lipoproteins, Kenneth R Feingold MD, Car Grunfeld, PhD, NCBI bookshelf).

“High Density Lipoproteins” (HDL) are the smallest of the lipoproteins (6-12.5 nm) (MW 175-500 kD) and most dense (around 1.12 g/ml). HDL contains several types of apolipoproteins including primarily apo-AI, II & IV, apo-CI, II and III, apo-D and apo-E. HDL contains approximately 35-55% protein, 3-15% triglycerides, 24-46% phospholipids, 15-30% cholesteryl esters and 2-10% cholesterol.

“Low Density Lipoproteins” (LDL) are cholesterol-rich lipoprotein with a density comprised between 1.019 and 1.063 g/mL and a diameter between 18 and 25 nm. LDL comprises apolipoprotein B-100.

LDL can be a native LDL, non-acetylated and non-hydroxylated or a modified LDL, hydroxydated and acetylated LDL.

Thus, the present invention relates to a complex formed between the compound of formula (I) or (II) as previously described and a High Density Lipoprotein (HDL), a Low Density Lipoprotein (LDL) or a modified Low Density Lipoprotein (LDL) such as oxidized LDL or acetylated LDL.

According to the present invention, “modified LDL” means oxidized or acetylated low-density lipoprotein (LDL). Modified low-density lipoproteins (LDL) are known by the skilled person to be recognized by the scavenger receptors of macrophages. Typically, they can be obtained by incubation in the presence of copper sulphate or a free radical generator (oxidized LDL) or by acetylation (acetylated LDL) (see A Modification Method for Isolation and Acetylation of Low Density Lipoprotein of Human Plasma by Density Discontinuons Gradient Ultracentrifugation, J. Z. Reza et al, Journal of Biological Sciences 10 (8): 785-789, 2010 ISSN 1727-3048).

The complex of a compound of formula (I) or (II) as previously described with a lipoprotein and in particular with HDL allows its uptake by tumor-associated macrophages, inducing a strong oxidative burst and an innate anti-cancer immune response. Thus, in a preferred embodiment, the invention relates to a complex formed between the compound of formula (I) or (II) as previously described and a HDL.

Typically, the lipoproteins are obtained from a biological sample and preferably from plasma of donors. In particular, said sample is normal or healthy plasma, preferably normolipidemic plasma. The methods suitable for obtaining, in particular separating and/or purifying the different fractions of lipoprotein are well known by the person skilled in the art (see Schumaker & Puppioe, 1986). Illustrative isolation methods include, but are not limited to ultracentrifugation, PEG precipitation, heparin MnCl2 precipitation, sodium phosphotungstate precipitation, dextran sulfate precipitation, gel filtration, fast protein liquid chromatography (FPLC) and immunoaffinity capture. Protocols for these and other HDL isolation methods are readily available. Thus, for example, illustrative, but non-limiting protocols for HDL isolation by PEG precipitation, heparin MnCl2 precipitation, sodium phosphotungstate precipitation, and dextran sulfate precipitation are described by Wieve and Smith, 1985.

Typically, the method for preparing the complex formed with a compound of formula (I) or (II) as previously described incorporated into a lipoprotein comprises the steps of contacting the compound of formula (I) or (II) as previously described with the lipoprotein and purifying said complex by dialysis. Then, the concentration of said complex can be determined by mass spectrometry.

The complex as previously defined can also comprise platinium compounds, preferably selected from the group consisting of cisplatin, carboplatin, oxaliplatin, tetraplatin, iproplatin, satraplatin, nedaplastin, laboplatin, picoplatin or ProLindac (polymere-platinate-DACH AP5346), preferably cisplatin and oxaliplatin.

According to the present invention “ProLindac” means a diaminocyclohexane (DACH)-platinum (Pt) complex coupled with hydroxypropyl methacrylamide (HPMA) copolymer (NCI Drug Dictionary, National Cancer Institute).

The present invention also relates to the complex as previously defined for its use as a medicament.

Method

The inventors showed that the compound of formula (I) and the compound of formula (II) are able to inhibit HSP70 activity. Thus, in a further aspect, the present invention also relates to the non-therapeutic use of the compound of formula (I), the compound of formula (II) or the complex as described above for inhibiting HSP70 activity and inducing cell apoptosis in vitro.

The present invention also relates to a method for inhibiting HSP70 protein by using:
the compound of formula (I) as previously defined,
the compound of formula (II) as previously defined, or
the complex as previously defined,
   in the presence of a HSP70 protein.

Pharmaceutical Composition T

he present invention also provides a pharmaceutical composition comprising, as active principle, the compound of formula (I) or (II) or the complex as previously defined and a pharmaceutically acceptable excipient.

The term “pharmaceutical composition” in the present invention refers to any composition comprising compound of formula (I) or the compound of formula (II) or the complex as previously defined and at least one pharmaceutically acceptable excipient.

By the term “pharmaceutically acceptable excipient”, it is herein understood a carrier medium which does not interfere with the effectiveness of the biological activity of the active ingredient(s) and which is not excessively toxic to the host at the concentration at which it is administered. Said excipients are selected, depending on the pharmaceutical form and the desired method of administration, from the usual excipients known by a person skilled in the art.

Formulation of a suitable composition can be carried out using standard pharmaceutical formulation chemistries and methodologies, all of which are readily available to the reasonably skilled artisan. For example, the compound of formula (I) or (II) or the complex as previously defined can be combined with one or more pharmaceutically acceptable excipients or vehicles. Auxiliary substances, such as wetting or emulsifying agents, pH buffering substances, reducing agents and the like, may be present in the excipient or vehicle. Suitable reducing agents include cysteine, thioglycerol, thioreducin, glutathione and the like. Excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

It is known from the prior art that HSP70 protein is involved in cancers. The inventors have found and demonstrated the inhibitory activity of compounds as previously defined towards HSP70 protein.

Accordingly, it will be acknowledged that the compounds of formula (I) or formula (II) as previously defined which are HSP70 protein inhibitors, the complex or the pharmaceutical composition as previously defined can be used as a medicament, preferably as a therapeutic agent for inhibiting the HSP70 protein activity, in particular for treating cancers of patients.

Also, it will be acknowledged that the compounds of formula (I) or formula (II) as previously defined which are HSP70 protein inhibitors, the complex or the pharmaceutical composition as previously defined can be used as a medicament, preferably as a therapeutic agent for inhibiting for inhibiting the HSP70 protein activity, in particular for treating tumors of patients.

Thus, the present invention also relates to the compound of formula (I) or (II), the complex or the pharmaceutical composition as previously defined for its use in the treatment of tumors and preferably solid tumors of a patient by inhibiting the HSP70 protein activity thereby inducing apoptosis of tumor cells and re-educating macrophages.

The present invention also relates to a method of treating cancers, comprising administering to a patient in need thereof, a therapeutically efficient amount of a compound of formula (I) or (II) or of the complex as previously defined, or of a pharmaceutical composition comprising said compound or said complex.

The present invention also relates to a method of treating tumors and preferably solid tumors of a patient, comprising administering to a patient in need thereof, a therapeutically efficient amount of a compound of formula (I) or (II) or of the complex as previously defined, or of a pharmaceutical composition comprising said compound or said complex.

As used herein, the term “subject” and “patient” which are used herein interchangeably refers to any member of the animal kingdom, preferably a mammal including human, pig, chimpanzee, dog, cat, cow, mouse, rabbit or rat. More preferably, the subject is a human, including for example a subject that has a tumor.

As used herein, the term “treatment”, “therapy”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease. In particular, with reference to the treatment of a cancer, the term “treatment” may refer to the inhibition of the growth of the tumor or the reduction of the size of the tumor.

In a particular embodiment, the patient suffers from a cancer and in particular a solid cancer selected from the group consisting of adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), sarcomas such as liposarcoma and soft-tissue sarcoma, brain and central nervous system cancer (e.g. meningioma, astocytoma, oligodendrogliomas, ependymoma, gliomas, medulloblastoma, ganglioglioma, germinoma, craniopharyngioma), breast cancer (e.g. ductal carcinoma in situ, infiltrating ductal carcinoma, infiltrating lobular carcinoma, lobular carcinoma in situ), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), kidney cancer (e.g. renal cell cancer), laryngeal and hypopharyngeal cancer, liver cancer, hepatic adenoma, focal nodular hyperplasia, hepatocellular carcinoma), lung cancer (e.g. small cell lung cancer, non-small cell lung cancer), mesothelioma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma).

By a “therapeutically efficient amount” is intended an amount of the compound of formula (I) or (II) or the complex as previously disclosed or a pharmaceutical composition administered to a subject that is sufficient to inhibit the HSP70 protein activity and induce tumor regression in the patient.

The compound of formula (I), the compound of formula (II), the complex or the pharmaceutical composition as previously described may be administered by any suitable route including topical administration or systemic administration such as enteral administration or parenteral. The compound of formula (I), the compound of formula (II), the complex or the pharmaceutical composition as previously described can be administered by intra-dermal, subcutaneous, percutaneous, intra-muscular, intra-arterial, intra-peritoneal, intra-articular, intra-osseous or other appropriate administration routes. In a preferred embodiment the compound of formula (I), the compound of formula (II), the complex or the pharmaceutical composition as previously described is administered by intra-venous infusion or intra-muscular administration route.

The compound of formula (I) or formula (II), the complex or the pharmaceutical composition as previously disclosed can be administrated in one or more doses. In another embodiment, said effective amount of the compound of formula (I) or formula (II), the complex or the pharmaceutical composition as previously disclosed are administrated as a single dose. In another embodiment, said effective amount of the compound of formula (I) or formula (II), the complex or the pharmaceutical composition as previously disclosed are administrated as more than one dose over a period time. Timing of administration is within the judgment of managing physician and depends on the clinical condition of the subject. Physiological data of the patient (e.g. age, size, and weight), the routes of administration and the disease to be treated have to be taken into account to determine the appropriate dosage.

The compound of formula (I) or formula (II), the complex or the pharmaceutical composition used according to the invention can be used alone or in combination with one or more other anti-cancer agent, surgery, immunotherapy and/or radiotherapy. The compound, complex as previously described and anti-cancer agent may be administered simultaneously or sequentially.

According to the present invention, immunotherapy is the treatment that stimulates the host immune system in a patient against malignant process and destroys cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. For example, the immune effector can be monoclonal antibodies (MAbs) that are covalently linked to cell-killing drugs. Alternatively, the effector may be a lymphocyte carrying a surface molecule that interacts, either directly or indirectly, with a tumor cell target. Various effector cells include cytotoxic T cells and NK cells. The immune effector may also be immune stimulating molecules such as IL-2, IL-4, IL-12, GM-CSF, gamma-IFN, chemokines, such as MIP-1, MCP-1, IL-8, and growth factors, such as FLT3 ligand or immune check point inhibitors such as anti-PD-1, anto-PDL-1 or anti-CTLA-4 antibodies. Immunotherapy may also include radioimmunotherapy, vaccines, and the like.

The anti-cancer agent is preferably a chemotherapeutic agent, such as for example: (i) an inhibitor of DNA replication like DNA binding agents, in particular alkylating or intercalating drugs, (ii) an antimetabolite agent such as DNA polymerase inhibitors or Topoisomerase I or II inhibitors, or (iii) an anti-mitogenic agent such as alkaloids. Such examples of chemotherapeutic agents include with no limitations: 5-FU, Oxaliplatin, Cisplatin, Carboplatin, Irinotecan, Cetuximab, Erlotinib, Docetaxel, and Paclitaxel.

The compound of formula (I) or formula (II) or the complex as previously described can be used as the sole active ingredient or in combination with one or more other active substances. The peptide and said one or more active substances may be administered simultaneously or sequentially. Said active substance is preferably an anti-cancer agent.

The invention also relates to a combination product (“kit of parts”) comprising firstly a compound of formula (I) or (II), the complex or the pharmaceutical composition as previously described and another anti-cancer agent well-known in the art for simultaneous combined use or time-shifted in a treatment of cancer, as defined above.

The present invention relates to a compound, a complex or pharmaceutical composition as previously described comprising said compound and an anti-cancer agent as a combined preparation for simultaneous, separate or sequential use for treating cancer in a subject.

Kit

The present invention also relates to a kit comprising a compound of formula (I) or formula (II), a complex or a pharmaceutical composition as described above.

Preferably, the present invention also relates to a kit comprising the compound of formula (I) or (II), the complex or the pharmaceutical composition as described above and an anti-cancer agent or a platinum compound as defined above.

In one embodiment, the kit also comprises an apparatus used for administering the compound of formula (I) or formula (II), the complex, the pharmaceutical composition as previously defined to a subject.

In one embodiment, the kit further comprises instructions for administering the compound of formula (I) or formula (II), the complex, the pharmaceutical composition as previously defined to a subject.

In one embodiment, the kit comprises an additional therapeutic agent, more particularly an anti-cancer agent. In one embodiment, the additional therapeutic agent is another agent for the treatment of tumor according to the invention.

In one embodiment, the additional therapeutic agent has a form suitable for the same route of administration as the compound of formula (I) or formula (II), the complex, the pharmaceutical composition of the invention.

In another embodiment, the additional therapeutic agent has a form suitable for a different route of administration to that of the compound of formula (I) or formula (II), the complex, the pharmaceutical composition according to the invention.

Although the exemplary embodiments of the present invention have been disclosed for illustrative purposes, a person skilled in the art will appreciate that various modifications, additions and substitutions are possible, without departing from the scope and spirit of the invention as disclosed in the accompanying claims.

The present invention will now be illustrated using the following examples and figures, which are given by way of illustration, and are in no way limiting.

### BRIEF DESCRIPTION OF DRAWINGS

Table 1. High throughput screening of chemical agonists of the A18 peptide aptamer. The screening was based on the ability of the molecules to inhibit the association A18/HSP70.

Table 2. Selected “Hits” with high inhibitory activity against A18 binding to HSP70. Their chemical structure is shown as well as their ability to block HSP70/A18 interaction (Biacore).

FIG. 1: Mouse embryonic fibroblasts (MEF) knock out for heat shock factor-1 (HSF1−/−) do not express HSP70. A western blot of inducible HSP70 and constitutive HSC70 is shown.

FIG. 2: Small molecules hits A, B and C inhibit HSP70 chaperone activity. A) Protein aggregation was determined in the presence or absence of recombinant HSP70 (10 ng). When indicated A18 or molecules A, B and C were added (1 μM). B) Protein aggregation was determined in the presence or absence of recombinant HSP70, HSC70 or HSP90 (10 ng).

When indicated A18 or molecules A, B and C were added (1 μM). A0, a non-relevant peptide aptamer was used here as a negative control. Values are normalized for each HSP. 100% chaperone effect being the inhibition of protein aggregation induced by addition of the recombinant chaperones.

FIG. 3. The chemical molecule hits sensitize cancer cells to cisplatin. A) Human cervix HeLa or B) mouse colorectal cancer CT-26 cells were incubated during 48 h with cisplatin (25 μM) in the presence of absence of molecules A, B and C (2 μM). Cell survival was measured by a colorimetric assay staining.

FIG. 4. The chemical molecules lose their chemo-sensitizing properties in cells not expressing HSP70. MEF HSF1−/− cells were incubated during 48 h with cisplatin (25 μM) in the presence of absence of molecules A, B and C (1 μM). Cell death was measured by a colorimetric assay staining.

FIG. 5: Chemical HSP70 inhibitors increase the number of apoptotic cells. Hela cells were treated with cisplatin (CDDP, 25 μM, 24 h) in the presence or absence of the chemical molecules A, B and C (2 μM). Apoptosis mas measured by flow cytometry (AnnexinV+/IP−).

FIG. 6: Molecule B-vectorized with HDL induces ROS from macrophages. A: LDL and HDL were purified by density gradient ultracentrifugation. Total cholesterol in lipoproteins was adjusted to 1 mM. Lipoproteins were then incubated with the HSP70 inhibitor Molecule B (100 μM) for 3 hours at 37° C., then submitted to dialysis (two times against 1L PBS, cutoff 8000 Da). Total molecule B concentration in lipoproteins was then measured by mass spectrometry. B: For macrophages activation, human M2 macrophages were treated for 2 hours with Molecule B (10 μM in DMSO) or vectorized in LDL or HDL (10 μM final Molecule B concentration). Percentage of ROS positive macrophages are represented as mean value +/− SEM. n=4, **: p<0.01, NS=Not Significant.

FIG. 7: HSP70 inhibitor vectorization in HDL prevents tumor growth by targeting macrophages. A: Balb-c mice were injected with CT-26 colorectal cancer cells (10⁶ cells/mice, s.c.). At the indicated times mice were treated either with PBS, or HDL-Molecule B (100 μM cholesterol, 10 μM Molecule B, 100 μl/mouse). n=4. B: Tumor volume was measured every 3 days, and represented as mean value +/− SEM, ***: p<0.001. C: Apoptosis and macrophage infiltration were t-determined in histological slides labeled with a cleaved caspase-3 antibody (Green) and a F4/80 antibody (Red), and with DAPI. Pictures were chosen in random fields and are representative of five pictures taken for each condition. n=4, scale bar=50 μm. D: ROS production in tumors was measured in histological slides by DAPI/DHE staining. Pictures, taken in random chosen fields, are representative of five pictures taken for each condition. n=4, scale bar=E, F & G: Quantifications of the immunofluorescence intensity of Cleaved Caspase-3 (E), F4/80 (F) and DHE (G). Data are represented as mean increase vs. CTL +/− SEM. n=4, *: p<0.05, ***: p<0.001, ****: p<0.0001.

FIG. 8: In vivo effects of cisplatin vectorization in LDL and Molecule B vectorization in HDL. A: Balb-c mice were injected with CT-26 colorectal cancer cells (10⁶ cells/mice, s.c.). At the indicated times mice were treated either with PBS, HDL-Molecule B (100 μM cholesterol, 10 μM Molecule B, 100 μ/mouse) or LDL-Cisplatin (100 μM cholesterol, 1.5 mg/kg cisplatin)+HDL-Molecule B (100 μM cholesterol, 10 μM Molecule B, 100 l/mouse). n=4 mice per groups. B: Tumor volume was measured every 3 days and represented as mean value+/−SEM. *: p<0.05. C: Apoptosis and macrophage infiltration were t-determined in histological slides labeled with a cleaved caspase-3 antibody (Green) and a F4/80 antibody (Red), and with DAPI. Pictures were chosen in random fields and are representative of five pictures taken for each condition. n=4, scale bar=50 μm. D: ROS production in tumors was measured in histological slides by DAPI/DHE staining. Pictures, taken in random chosen fields, are representative of five pictures taken for each condition. n=4, scale bar=50 μm. E, F & G: Quantifications of the immune-fluorescence intensity of Cleaved Caspase-3 (E), F4/80 (F) and DHE (G). Data are represented as mean increase vs. CTL+/−SEM. n=4, *: p<0.05, ****: p<0.0001.

### EXAMPLES

### Material and Methods

High Throughput Screening of Chemical Agonists of A18 Peptide Aptamer.

A high-throughput screening assay, AptaScreen™ (developed by Aptanomics SA; (Baines I C et al. Drug Discov Today. 2006;11:334-341; Rerole A L, et al. Cancer Res 20119;71:484-495)) was performed by the Company Imaxio (Lyon, France) on a library of almost 60,000 small molecules. The AptaScreen™ assay is based on an automated dual luminescence (luc and ruc reporter genes) yeast two-hybrid assay, with HSP70 expressed as a ‘bait’ and A18 aptamer expressed as a ‘prey’. The HSP70/A18 interaction directs the transcription of the luc reporter gene, while in the same assay a control protein/aptamer couple directs the transcription of the ruc reporter. A small molecule can be considered as ‘hit candidate’ when it inhibits the interaction between HSP70 and A18 (i.e. decreases the luciferase signal), but not the control interaction (i.e. no or little change in the ruc signal). Molecules were screened at a concentration of 10 μM and then hit candidates were confirmed by dose-response assays, following the standard protocol (Rerole A L, et al. Cancer Res 20119;71:484-495).

Cells, Plasmids and Transfections. HeLa cells, HSF1−/− MEFs, and CT-26 cells were grown as monolayers in a controlled atmosphere (37° C., 5% CO2) using RPMI 1640 medium or DMEM glucose 4.5 g. L-1 medium supplemented with 10% (v/v) fetal bovine serum (FBS) (all mediums and FBS come from Lonza, Switzerland). HSF1−/− MEFs were transiently transfected with HSP70 cloned into HA-tagged pcDNA3.1 vector or co-transfected with HSP70 and with A18 peptide aptamer, or A14 as control aptamer, cloned into Myc/6His-tagged pcDNA3.1, or with the empty vector as control.

HSP70 Chaperone Activity Study In Vitro.

HSP70 chaperone activity was evaluated with a protein thermolability assay. HSF1−/− MEFs extracts were incubated 20 minutes on ice in lysis buffer (50 mM HEPES, 150 mM NaCl, 5 mM EDTA, 0.1% NP40) supplemented with protease inhibitors (Roche, France). Supernatants were recovered after g×14,000 centrifugation at 4° C., during 15 minutes, and a protein concentration assay was performed (Dc Assay Kits, Bio-Rad, France) against bovine serum albumin (BSA) standard range.

Cellular extracts, to which recombinant HSP70, HSP90 or HSC70 were added with or without the small molecules to test, were diluted to a final concentration of 2 mg. mL-1 in pH 7 Tris-HCl buffer and heated at 55° C. during 1 hour. After g−16,000 centrifugation at 4° C. during 10 minutes, supernatant native protein quantity was determined by Lowry method (Dc Assay Kits, Bio-Rad). This final protein concentration was then compared to the initial protein concentration in supernatants to quantify denatured proteins.

Cell Death Analysis.

3.5×10⁴ adherent cells were plated onto 24-well culture plates in complete medium. Cells were treated with small molecules at various concentrations (1 μM to 30 μM) for 4 hours to determine cellular IC50. Then, cells of half of each 24-well culture plates were treated with cisplatin (CDDP, 25 μM) for 48 hours. Cell death was measured by the crystal violet colorimetric assay staining.

Lipoprotein Purification

HDL were purified from non-therapeutic healthy plasma (EFS Besançon, France) by density gradient ultracentrifugation as described previously by Redgrave et al. 1975. Anal Biochem.; 65(1-2):42-9). Briefly, plasma density was adjusted to 1.21 by the addition of KBr salt (Sigma Aldrich, #746444), 5 ml of plasma was then transferred to a 13.2 ml Ultra-Clear centrifuge tube (Beckman Coulter, 344059). 3m1 of 1.063 density KBr solution (ddw, 0.1 g/l EDTA, 0.02 g/l Sodium Azide), then 2 ml 1.019 density KBr solution (ddw, 0.1 g/1 EDTA, 0.02 g/1 Sodium Azide) and finally 2 ml of 1.000 density NaCl solution (ddw, 0.1 g/l EDTA, 0.02 g/l Sodium Azide) were gently layered on top of the plasma. Samples were then centrifuged for 24 hours at 40.000 rpm on a Beckmann ultracentrifuge (XXL-80) equipped with a swinging rotor SW 41 TI, with low acceleration and no brake. After centrifugation, the HDL fraction was collected at the interface of the 1.063 and 1.21 solutions. Total cholesterol was dosed using the Cholesterol Quantitation kit (CliniSciences, JM-K603-100) as indicated by the manufacturer's instructions, and the final cholesterol concentration was adjusted to 1mM in each sample by the addition of PBS.

Molecule B Incorporation in Lipoproteins.

Molecule B was first diluted in pure DMSO to a final concentration of 100 μM, then diluted 10 times in 1 mM HDL fractions and incubated 4 hours at 37° C. Unbound molecule B and DMSO were then removed by two successive dialyses using Spectrum™ Spectra/Por™ 1 RC Dialysis Membrane Tubing (6000 to 8000 Dalton Cut Off, Fisher Scientific, 08-670C), against 1000 times the volume of PBS. Molecule B incorporation HDL was then assessed by mass spectrometry.

Cell Culture

Human macrophages were differentiated from peripheral blood monocytes obtained from Buffy Coats of healthy donors (EFS Besancon, France). Briefly, to extract monocytes, 15 ml of blood (diluted 2-times in PBS) was gently layered on a 46-65% Percoll gradient solution (Sigma Aldrich P1644-1L), and centrifuged for 30 min at 550 g, RT. After centrifugation, the upper ring containing monocytes was recovered and seeded on 12-well plates (5.10⁵ monocytes per well) in RPMI 10%FBS, 100 UI/ml PSA, 37° C. 5% CO₂ and differentiated into macrophages by stimulating cells 7 days with M-CSF (100 ng/ml, Miltenyi Biotechnology #130-095-372).

Flow Cytometry Analysis

For macrophage ROS production analysis by flow cytometry, cells were cultured for 30 min at 37° C. and 5% CO₂, in DHE (10 μM in PBS), scraped out and centrifuged (10 min, 1500 rpm, 4° C.). Cells were fixed for 5 min in a PBS 4% PFA solution and analyzed using an LSRII flow cytometer (Becton Dickinson). Primary Size-Granularity dot plot allowed us to discriminate cells from debris, and DHE positive cells were obtained by comparing red fluorescence vs. unstained samples.

Mouse Procedures

6-8 week-old female Balb/c mice were purchased from Charles River. The Balb/c-derived mouse colon carcinoma cell line CT26 (CRL2638™) was purchased from American Type Culture Collection (ATCC), and cultivated according to the manufacturer's instruction. CT26 cells (10⁶ cells/mice) were injected subcutaneously in the left flank. Time 0 was considered when the size of the tumor-reached 6 mm³. For tumor growth experiments, mice were treated i.p. at day 7, 14 and 21, with PBS, HDL-Molecule B (100 μM cholesterol, 10 μM Molecule B, 100 μl mouse) and euthanized at on day 25 (n=5 mice per groups). Tumors were measured every three days with a digital caliper (tumor volume was determined using the ½×Length×Width² formula). Experiments were approved by the ethical comity of the Université de Bourgogne (protocol N3613).

### Results

### Screening of a Chemical Library against the A18 Aptamer—HSP70 interaction.

A high-throughput screening assay, AptaScreen™ (developed by Aptanomics SA; ^{22,23}), was performed by the Company Imaxio (Lyon, France) on a library of almost 60,000 small molecules including most of the marketed drugs. The AptaScreen™ assay is based on an automated dual luminescence (luc and ruc reporter genes) yeast two-hybrid assay, with HSP70 expressed as a ‘bait’ and a peptide aptamer (A18), the inventors previously isolated that bound to the ATP domain of HSP70 expressed as a ‘prey’ (Rerole A L, et al. Cancer Res 20119;71:484-495). A small molecule can be considered as ‘hit candidate’ when it inhibits the interaction between HSP70 and A18 (i.e. decreases the luciferase signal). Molecules were screened at a concentration of 10 μM. Eight molecules (hit candidates) were initially identified. From the dose-response studies, three of them were retained on the basis of their specificity and high inhibitory activity against A18 binding to HSP70 (Table 1 and 2). Interestingly, molecules 2 and 3 are analogues.

TABLE 1

| | Screening | Confirmation | IC50 | HSP70 |
|---|---|---|---|---|
| Molecule | inhibition | inhibition | (μM) | specificity |
| A | 105 | 34 | 19.6 | YES |
| B | 105 | 59 | 0.2 | YES |
| C | 103 | 61 | 0.46 | YES |

TABLE 2

| | | Activity profil/50% |
|---|---|---|
| | Name, formula and structure | effective concentration |
| A | 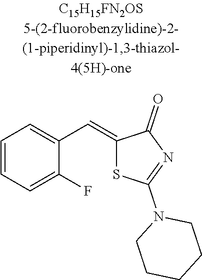 [SMILES: O=C1N=C(N2CCCCC2)S/C1=C\c1ccccc1F] | EC₅₀ = 19.6 μM |
| B | 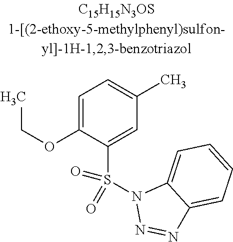 [SMILES: CCOc1ccc(C)cc1S(=O)(=O)n1nnc2ccccc21] | EC₅₀ = 0.2 μM |
| C | 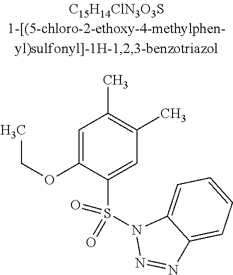 [SMILES: CCOc1cc(C)c(C)cc1S(=O)(=O)n1nnc2ccccc21] | EC₅₀ = 0.46 μM |

The Small Molecule Hit-Candidates Inhibits HSP70 Chaperone Activity In Vitro.

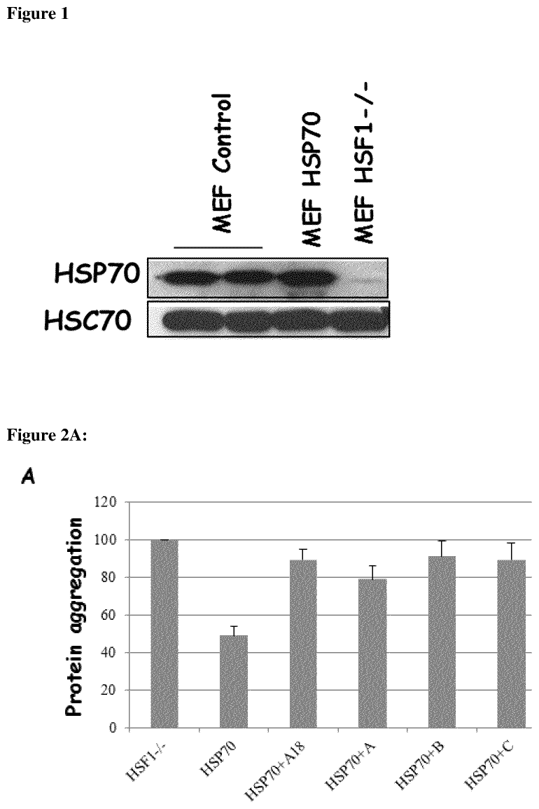

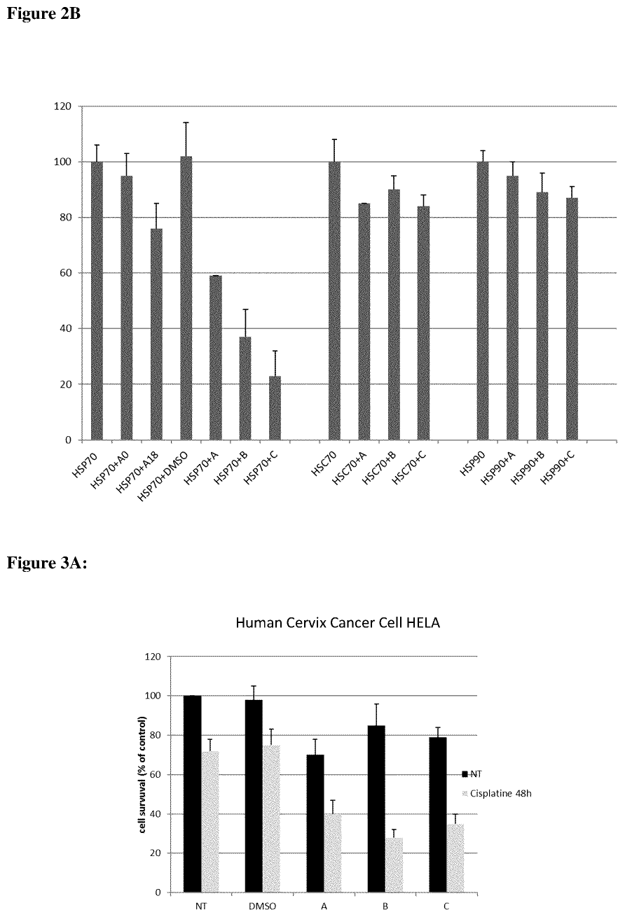

A method to study the chaperone activity of HSPs was set up. Proteins were extracted from mouse embryonic HSF1−/− cells and they were heat shocked. HSF1 is the main transcription factor responsible of HSP expression after a stress. Therefore, this genetic background allowed us to work with reduced contamination with endogenous inducible HSPs like HSP70 (FIG. 1). An equal amount of proteins was submitted to a heat shock (55° C., 1 h) and the percentage of protein aggregation (which is directly related to the amount of denatured proteins) was determined in the presence or absence of recombinant HSP70, with or without the chemical molecules to be tested. As shown in FIG. 2A, HSP70 was able to reduce the amount of aggregated proteins in a very significant manner, demonstrating its chaperone function. A similar effect was observed when adding HSC70 or HSP90. As expected, when the inventors added the A18 aptamer, a strong inhibition of HSP70 chaperone activity was observed that was not observed with a control aptamer (A0). This inhibitory effect was specific because A18 was unable to block recombinant HSC70 or HSP90 chaperone activity. All four small molecule hits inhibited HSP70 chaperone activity, albeit to a different extent. Molecule A only partially blocked HSP70 chaperone function but this effect seemed specific. Molecules B and C provoked a much more important inhibition (FIG. 2B).

Chemo-Sensitizing Properties of the Chemical Molecules Targeting HSP70

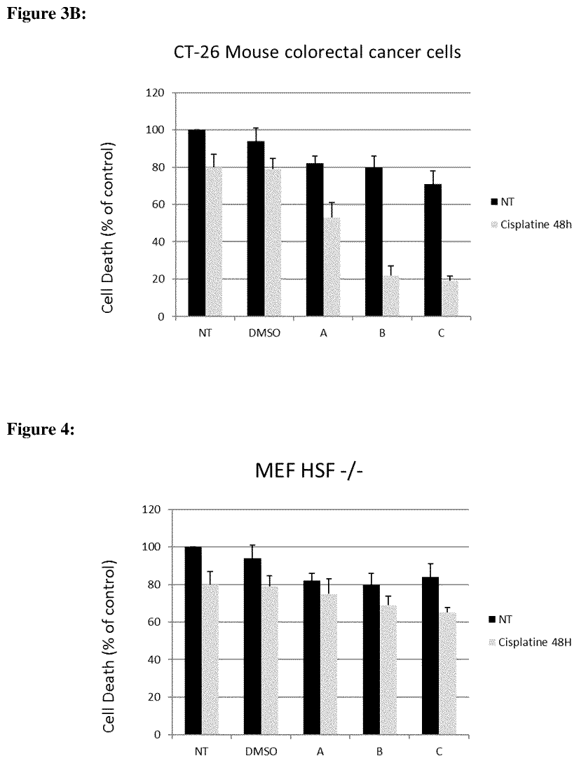

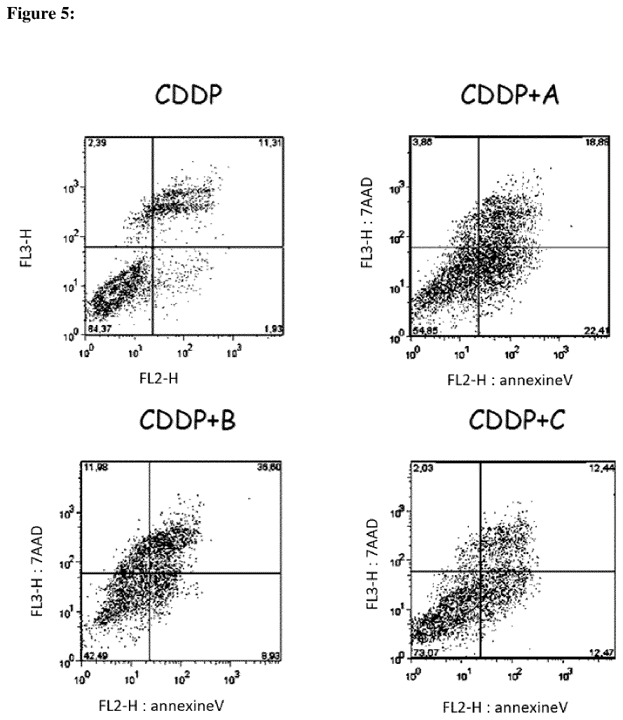

To study the effect of the molecules in cancer cells, the inventors used two different cancer cells lines: Human cervix cancer Hela cells and mouse colorectal cancer CT-26. Cells were treated with the small molecule hit (2 μM) either alone or together with cisplatin for 48 hours and cell survival was determined. As shown in FIGS. 3A and 3B, the molecules synergistically increased cell death induced by cisplatin, the most important effect been found for the molecule B. This cell death was associated with the inhibition of HSP70 because the molecules had hardly any effect on the MEF HSF1−/− cells (FIG. 4) which, as shown in FIG. 1, do not express inducible HSP70. Cells seem to die mainly by apoptosis, as detected by the amount of Annexin V positive/propidium iodide negative cells obtained after treatment with the molecule (FIG. 5). The inventors conclude that the hit candidates block HSP70 chaperone activity and sensitize cancer cells to apoptosis induced by cisplatin. Since throughout these results the molecule B seemed, in terms of specificity (IC50) and effect, the most promising, the inventors selected this molecule for the studies in vivo.

Molecule B Displays an Anti-Tumor effect in Mice bearing a Colorectal Cancer, which Involve Cytotoxic Macrophages

To study the molecule B in vivo, the inventors decided to vectorize the molecule with lipoproteins of high density (HDL) because these natural nano-vectors have been reported to solve solubility problems in hydrophobic and hardly soluble molecules (like all four molecules selected here including the molecule B) and to favor cellular uptake.

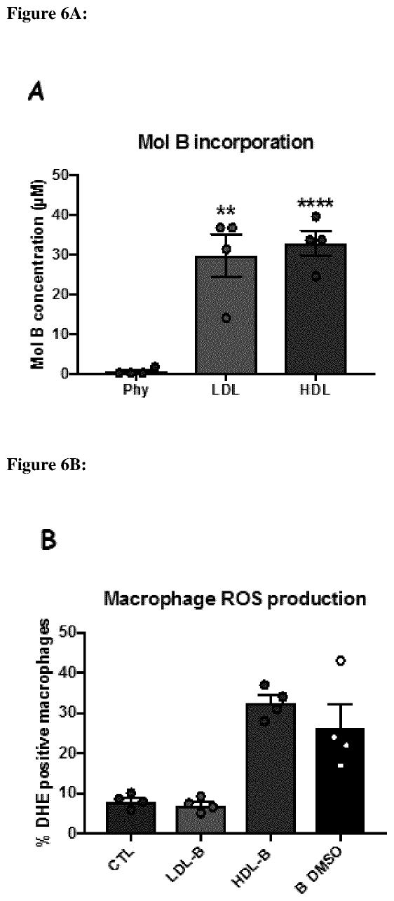

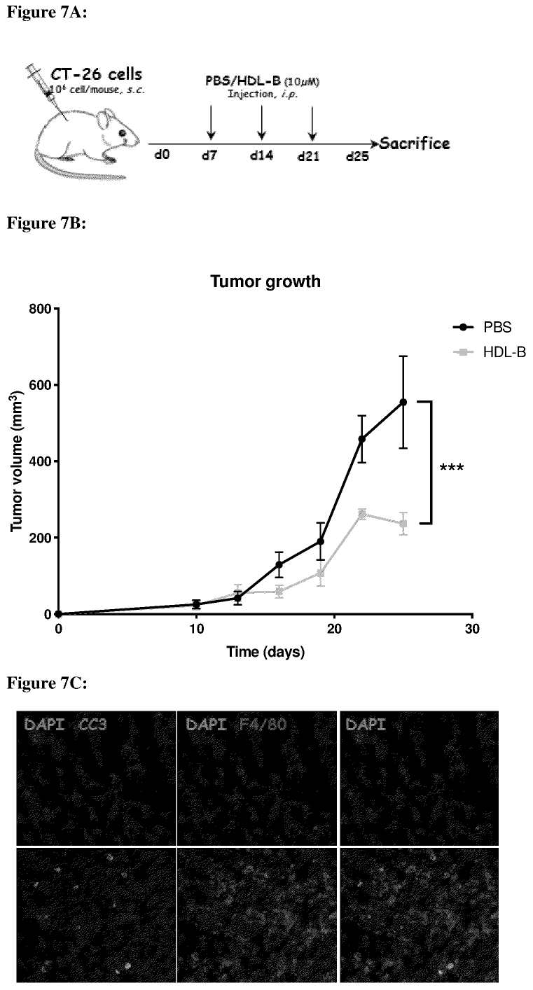

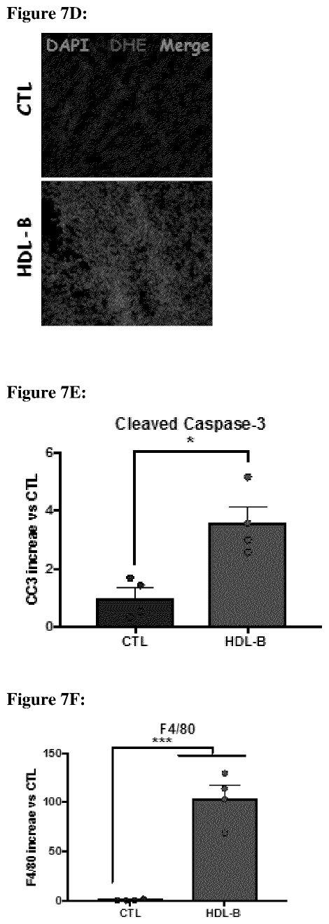

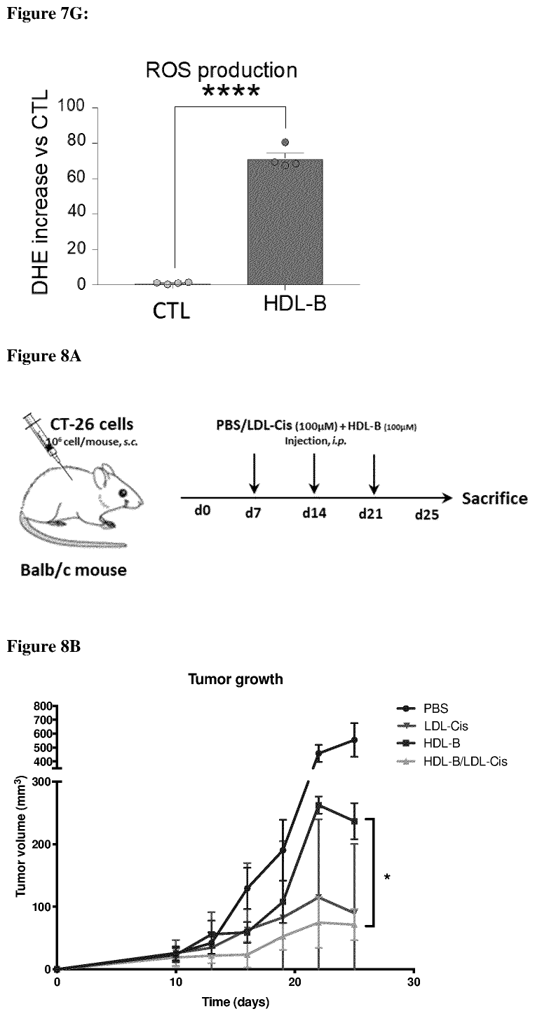

A syngeneic model in which mouse colon cancer CT-26 cells were injected into Balb/c mice was used. When tumor size reached about 0.9 mm³, mice were treated with the molecule B complexed to HDL (FIG. 6A) at 5 mg/kg (average concentration described in the literature for similar small chemical compounds), which was given i.p. every three days until the end of the experiment (determined for ethical reasons by the size of the tumor in the control group) (FIG. 7A). Treatment by compound B induced a decrease in tumor growth of 60% (FIG. 7B). In accord with the previous results, this tumor regression effect was associated with an increase in tumor cells apoptosis (caspase-3 activation. FIG. 7C, E). But the most remarkable effect induced by the treatment with the vectorized molecule B was a strong accumulation within the regressing tumor of macrophages with a cytotoxic (anti-tumor) phenotype (FIG. 7C-F), as shown by their ability to produce reactive oxygen species (ROS) (FIG. 7C-G).

This effect of the-molecule B favoring cytotoxic macrophages was confirmed in vitro, in macrophages isolated from buffy coats. HDL-molecule B was able to induced ROS production (FIG. 6B) whereas the same amount of molecule complexed to LDL did not have any effect, indicating the importance of the lipoprotein used as a nano-vector.

This molecule, the first described experimental therapeutic HSP70 inhibitor targeting macrophages in vitro and in vivo, may pave the way to a new type of immunotherapeutic molecules against chemo resistant cancers.

Combinational Effect of Molecule B-HDL and Cisplatin-LDL Complexes

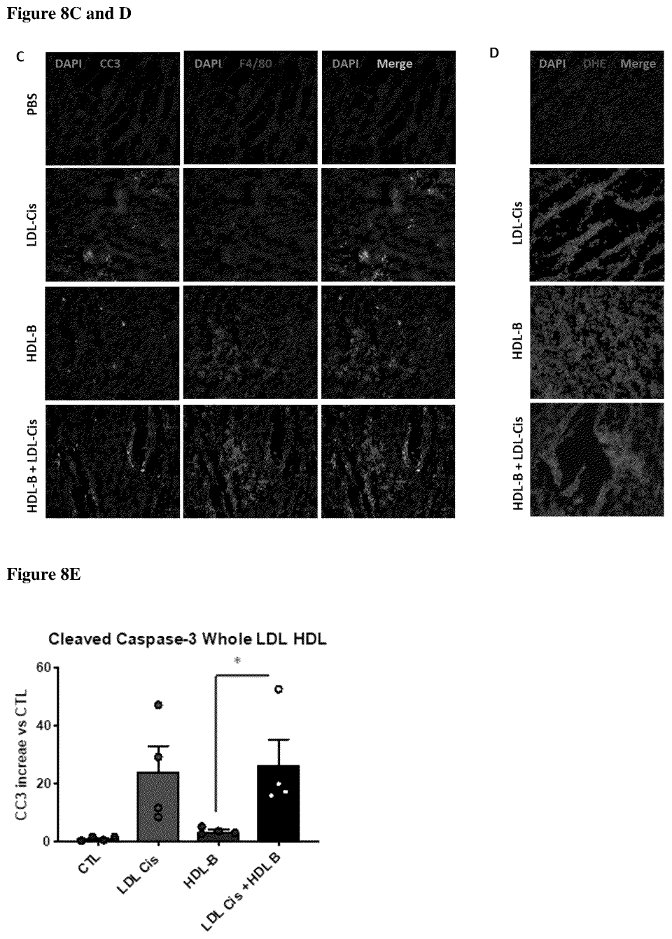

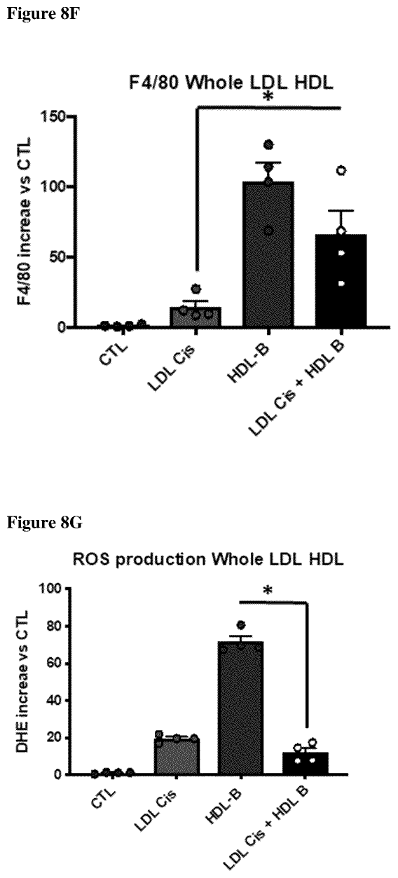

Finally, the inventors tested the impact of the association of cisplatin-LDL complexes together with molecule B-HDL complexes. LDL were purified by density gradient ultracentrifugation from buffy coats and incubated with cisplatin (to a final concentration of 1 mg/ml) for 4 hours at 37° C. Mice bearing CT-26 tumors were treated with LDL-Cisplatin alone, HDL-molecule B alone or the combination of both. The inventors observed a stronger decrease in tumor growth when using the combinational therapy (FIGS. 8A and B) Immunofluorescent staining revealed i) a strong induction of cancer cell apoptosis (FIGS. 8C and E), comparable to that observed in the animals treated with LDL-Cisplatin alone ii) a strong burst in macrophage infiltration comparable to that observed with the HDL-molecule B alone (FIGS. 8C and F). of note, a milder induction of ROS production was observed in this context (FIGS. 8D and G), probably due to a more advanced tumor regression in response to the combined treatment. This suggest that this combined strategy aiming to simultaneous target cancer cells with one drug (cisplatin-LDL) and tumor-infiltrating macrophages with the other (molecule B-HDL) allows a complementary additive effect, with no apparent increase in the drugs side-effects toxicity -as determined by the unchanged weight of the animals and the absence of apoptosis in the animals' epithelial cells in the intestinal crypts (data not shown).

Discussion

In this work, the inventors have identified small chemical molecules, agonists of the peptide aptamer A18. A18 is a thioredoxin-based aptamer with a 13 aminoacid variable region that binds to the ATP domain of HSP70 (Rerole A L, et al. Cancer Res 20119;71:484-495). The hits interfere with the chaperone activity of HSP70 in vitro, thus strongly suggesting that, as A18, they bind to the ATP domain of HSP70.

The four “drug candidates” described here sensitize cancer cells to death induced by cisplatin. Macrophages are essential component of the anti-cancer immune response and the inventors and others have reported a role for HSP70 in macrophages differentiation/maturation (Vega V L, et al. J Immnuol 2008;180:4299-307). Confirming these results, in the present application, the inventors have demonstrated that the i.p. injection of the molecule B in syngeneic mice bearing a tumor induced tumor regression that was associated with an impressive accumulation within the tumor of inflammatory cytotoxic macrophages (M1-like). Interestingly, to avoid problems of solubility, in these in vivo experiments the inventors vectorized the molecule by complexing it to natural HDL. The fact that the vectorization with HDL, but not with LDL, favors the effect of the molecule B in cultured macrophages suggests the importance of the nano-vector used and how the macrophages uptake of the HDL-molecule B complexes may involve specific receptors.

Cancer cells must extensively rewire their metabolic and signal transduction pathways, thereby becoming dependent on proteins that are dispensable for the survival of normal cells.

This HSPs-addition is the basis for the use of inhibitors of HSPs in cancer therapy. Today, with the exception of an inhibitor of HSP27 (an oligonucleotide antisense) all inhibitors in advanced clinical trials target HSP90 with somehow deceiving results and often unaccepted toxicity, which may be why they induce HSP70 expression that by its strong cell survival properties may counteract the efficacy of the HSP90 inhibitors. HSP70 can be considered as a protein that although it is not an oncogene, its presence is indispensable for the survival of cancer cells. HSP70 has a well-demonstrated role in apoptosis inhibition and autophagic cell death. Unfortunately, to date only a limited number of compounds that specifically target HSP70 have been identified. Leu et al described that the 2-phenylethynesulfonamide (PES), also known as pifithrin-α and originally identified as a molecule that interferes with the P53-induced apoptosis, specifically associated to the peptide binding domain of HSP70, induced autophagic cell death in cancer cells (but not apoptosis) and, in intraperitoneal administration, was able to inhibit the development of lymphomas in mice (Leu J I, et al. Mol Cell. 2009;36:15-27).

### CITED REFERENCES

1. Liu X, Kim C N, Yang J, Jemmerson R, Wang X. Induction of apoptotic program in cell-free extracts: requirement for dATP and cytochrome c. Cell. 1996;86:147-157.

2. Ferri K F, Kroemer G. Organelle-specific initiation of cell death pathways. Nat Cell Biol. 2001;3:E255-263.

3. Hu Y, Benedict M A, Ding L, Nunez G. Role of cytochrome c and dATP/ATP hydrolysis in Apaf-1-mediated caspase-9 activation and apoptosis. Embo J. 1999;18:3586-3595.

4. Li P, Nijhawan D, Budihardjo I, et al. Cytochrome c and dATP-dependent formation of Apaf-1/caspase-9 complex initiates an apoptotic protease cascade. Cell. 1997;91:479-489.

5. Zou H, Li Y, Liu X, Wang X. An APAF-1.cytochrome c multimeric complex is a functional apoptosome that activates procaspase-9. J Biol Chem. 1999;274:11549-11556.

6. Saleh A, Srinivasula S M, Acharya S, Fishel R, Alnemri E S. Cytochrome c and dATP-mediated oligomerization of Apaf-1 is a prerequisite for procaspase-9 activation. J Biol Chem. 1999;274:17941-17945.

7. Daugas E, Susin S A, Zamzami N, et al. Mitochondrio-nuclear translocation of AIF in apoptosis and necrosis. Faseb J. 2000;14:729-739.

8. Susin S A, Lorenzo H K, Zamzami N, et al. Molecular characterization of mitochondrial apoptosis-inducing factor. Nature. 1999;397:441-446.

9. Beckmann R P, Mizzen L E, Welch W J. Interaction of Hsp 70 with newly synthesized proteins: implications for protein folding and assembly. Science. 1990;248:850-854.

10. Murakami H, Pain D, Blobel G. 70-kD heat shock-related protein is one of at least two distinct cytosolic factors stimulating protein import into mitochondria. J Cell Biol. 1988;107:2051-2057.

11. Shi Y, Thomas J O. The transport of proteins into the nucleus requires the 70-kilodalton heat shock protein or its cytosolic cognate. Mol Cell Biol. 1992;12:2186-2192.

12. Schmitt E, Parcellier A, Gurbuxani S, et al. Chemosensitization by a non-apoptogenic heat shock protein 70-binding apoptosis-inducing factor mutant. Cancer Res. 2003;63:8233-8240.

13. Parcellier A, Gurbuxani S, Schmitt E, Solary E, Garrido C. Heat shock proteins, cellular chaperones that modulate mitochondrial cell death pathways. Biochem Biophys Res Commun. 2003;304:505-512.

14. Conroy S E, Latchman D S. Do heat shock proteins have a role in breast cancer? Br J Cancer. 1996;74:717-721.

15. Fuller K J, Issels R D, Slosman D O, Guillet J G, Soussi T, Polla B S. Cancer and the heat shock response. Eur J Cancer. 1994;30A:1884-1891.

16. Brondani Da Rocha A, Regner A, Grivicich I, et al. Radioresistance is associated to increased Hsp70 content in human glioblastoma cell lines. Int J Oncol. 2004;25:777-785.

17. Vargas-Roig L M, Gago F E, Tello O, Aznar J C, Ciocca D R. Heat shock protein expression and drug resistance in breast cancer patients treated with induction chemotherapy. Int J Cancer. 1998;79:468-475.

18. Nanbu K, Konishi I, Mandai M, et al. Prognostic significance of heat shock proteins HSP70 and HSP90 in endometrial carcinomas. Cancer Detect Prey. 1998;22:549-555.

19. Gurbuxani S, Bruey J M, Fromentin A, et al. Selective depletion of inducible HSP70 enhances immunogenicity of rat colon cancer cells. Oncogene. 2001;20:7478-7485.

20. Nylandsted J, Rohde M, Brand K, Bastholm L, Elling F, Jaattela M. Selective depletion of heat shock protein 70 (Hsp70) activates a tumor-specific death program that is independent of caspases and bypasses Bcl-2. Proc Natl Acad Sci USA. 2000;97:7871-7876.

21. Schmitt E, Maingret L, Puig P E, et al. Heat shock protein 70 neutralization exerts potent antitumor effects in animal models of colon cancer and melanoma. Cancer Res. 2006;66:4191-4197.

22. Baines IC, Colas P. Peptide aptamers as guides for small-molecule drug discovery. Drug Discov Today. 2006;11:334-341.

23. Rerole A L, Gobbo J, Schmitt E, Pais de Barros J P, Lanneau D, De Thonel A, Hammann A, Fourmaux E, Delidov O, Micjeau O, Lagrost L, Colas P, Kroemer G, Garrido C. Peptides and aptamers targeting HSP70: A novel approach for anti-cancer therapy. Cancer Res 20119;71:484-495.

24. Vega V L, Rodriguez-Silva M, Frey T, Gehrmann M, Diaz J C, Steinem C, Multhoff G, Arispe N, De Maio A. Hsp70 translocate into the plasma membrane after stress and is released into the extracellular environment in a membrane-associated form that activates macrophages. J Immnuol 2008;180:4299-307.

25. Leu J I, Pimkina J, Frank A, Murphy M E, George D L. A small molecule inhibitor of inducible heat shock protein 70. Mol Cell. 2009;36:15-27.

1. A method of treating tumors in a patient in need thereof by inhibiting HSP70 protein activity thereby inducing apoptosis of tumor cells and re-educating macrophages, comprising administering to the patient a therapeutically efficient amount of a compound of formula (I) or (II) 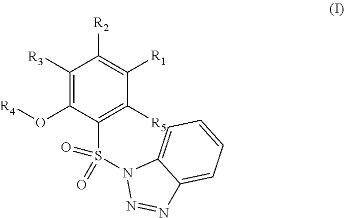
wherein R₁ represents a hydrogen atom, an halogen atom, a C1-C8 alkyl group,
R₂ represents a hydrogen atom, an halogen atom, a C1-C8 alkyl group,
R₃ represents a hydrogen atom, an halogen atom, a C1-C8 alkyl group,
R₄ represents a C1-C8 alkyl group, and
R₅ represents a hydrogen atom, an halogen atom, or a C1-C8 alkyl group, 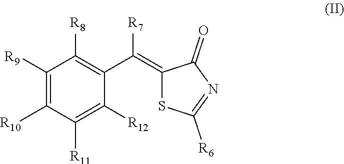
wherein R₆ represents an heterocycle preferably a piperidinyl group,
R₇ represents a hydrogen atom or a halogen atom,
R₈ represents a hydrogen atom or a halogen atom,
R₉ represents a hydrogen atom or a halogen atom,
R₁₀ represents a hydrogen atom or a halogen atom,
R₁₁ represents a hydrogen atom or a halogen atom, and
R₁₂ represents a hydrogen atom or a halogen atom.

2. The method according to claim 1, wherein the compound of formula (I) is chosen from the group consisting of a compound of formula (B) 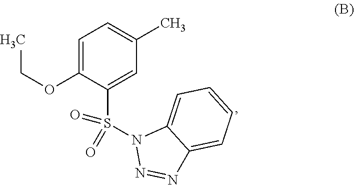 [SMILES: CCOc1ccc(C)cc1S(=O)(=O)n1nnc2ccccc21]
and
a compound of formula (C) 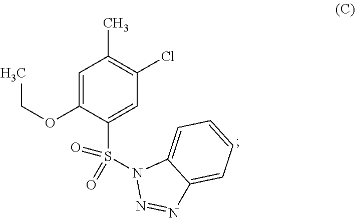 [SMILES: CCOc1cc(C)c(Cl)cc1S(=O)(=O)n1nnc2ccccc21]
and
the compound of formula (II) is a compound of formula (A)

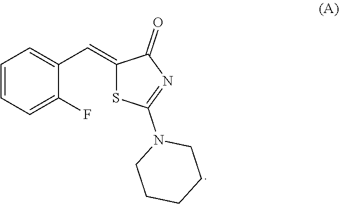 [SMILES: O=C1N=C(N2CCCCC2)S/C1=C\c1ccccc1F]

3. Complex formed between a compound of formula (I) or (II) and a lipoprotein, 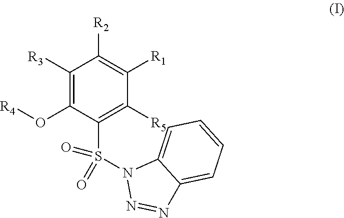
wherein R₁ represents a hydrogen atom, an halogen atom, a C1-C8 alkyl group
R² represents a hydrogen atom, an halogen atom, a C1-C8 alkyl group,
R₃ represents a hydrogen atom, an halogen atom, a C1-C8 alkyl group,
R₄ represents a C1-C8 alkyl group, and
R₅ represents a hydrogen atom, an halogen atom, or a C1-C8 alkyl group; 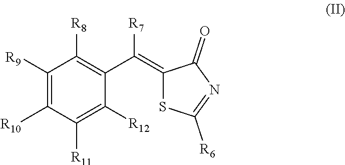
wherein R₆ represents an heterocycle preferably a piperidinyl group,
R₇ represents a hydrogen atom or a halogen atom,
R₈ represents a hydrogen atom or a halogen atom,
R₉ represents a hydrogen atom or a halogen atom,
R₁₀ represents a hydrogen atom or a halogen atom,
R₁₁ represents a hydrogen atom or a halogen atom, and
R₁₂ represents a hydrogen atom or a halogen atom.

4. The complex according to claim 3, wherein the compound of formula (I) is chosen from the group consisting of
a compound of formula (B) 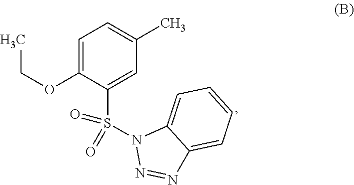 [SMILES: CCOc1ccc(C)cc1S(=O)(=O)n1nnc2ccccc21]
and
a compound of formula (C) 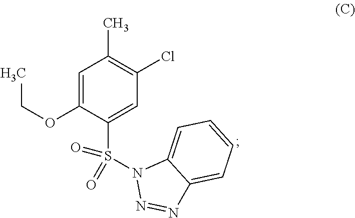 [SMILES: CCOc1cc(C)c(Cl)cc1S(=O)(=O)n1nnc2ccccc21]
and
the compound of formula (II) is a compound of formula (A)

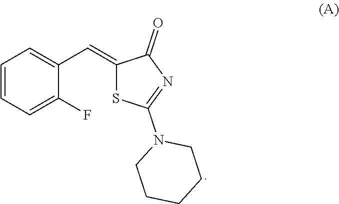 [SMILES: O=C1N=C(N2CCCCC2)S/C1=C\c1ccccc1F]

5. Complex as defined in claim 3, further comprising a platinum compound selected from the group consisting of cisplatin, carboplatin, oxaliplatin, tetraplatin, iproplatin, satraplatin, nedaplatin, lobaplatin, picoplatin and ProLindac (polymere-platinate-DACH AP5346).

6. (canceled)

7. Pharmaceutical composition comprising, as active principle, the compound of formula (I) or (II) as defined in claim 1 or a complex formed between the compound and a lipoprotein, and a pharmaceutically acceptable excipient.

8. A method of treating tumors in a patient in need thereof by inhibiting the HSP70 protein activity, thereby inducing apoptosis of tumor cells and re-educating macrophages, comprising administering to the patient a therapeutically effective amount of comprising the complex.

9. A method of treating cancer in a patient in need thereof comprising administering to the patient a therapeutically effective amount of a combination of a compound of formula (I) or (II) as defined in claim 1, a complex formed between the compound of formula (I) or (II) and a lipoprotein or a pharmaceutical composition comprising the compound or the complex, and one or more anti-cancer agents, surgery, immunotherapy and/or radiotherapy.

10. (canceled)

11. Kit comprising, the complex of claim 3 and an anti-cancer agent and/or a platinum compound.

12. The method of claim 1, wherein the tumors are solid tumors.

13. The method of claim 1, wherein one or more of R₁, R₂, R₃, R₄ and R₅ is a C1-C3 alkyl group.

14. The method of claim 1, wherein R₆ is a piperidinyl group.

15. The complex of claim 3, wherein the lipoprotein is a High Density Lipoprotein (HDL).

16. The method of claim 3, wherein one or more of R₁, R₂, R₃, R₄ and R₅ is a C1-C3 alkyl group.

17. The method of claim 3, wherein R₆ is a piperidinyl group.

18. The method of claim 8, wherein the tumors are solid tumors.
